(19) United States
(12) Patent Application Publication
(10) Pub. No.: US 2022/0087801 A1
(43) Pub. Date: Mar. 24, 2022

(54) CONTAMINANT SCAVAGING SYSTEM, APPARATUS, AND METHOD

(71) Applicants: James DiMarino, Naples, FL (US); Richard Rosenthal, Philadelphia, PA (US)

(72) Inventors: James DiMarino, Naples, FL (US); Richard Rosenthal, Philadelphia, PA (US)

(21) Appl. No.: 17/319,046

(22) Filed: May 12, 2021

Related U.S. Application Data
(60) Provisional application No. 63/101,770, filed on May 13, 2020.

(51) Int. Cl. A61C 17/12; A61L 9/20; A61L 9/14; A61L 9/015

(52) U.S. Cl. CPC A61C 17/12; A61L 9/015; A61L 9/14; A61L 9/20

(57) ABSTRACT

The present invention allows dental professionals to more efficiently and effectively control contaminants not only at the source of their creation (the mouth, for example), with optional configurations that comprise tiered guarding layers to protect the patient, the dentist, the dental assistant, the dental hygienist, as well as other dental professionals/staff, other patients, and subsequent patients to be treated in that operatory.

This application claims the benefit of U.S. Provisional Patent Application No. 63/101,770.

### BACKGROUND SECTION

There are several fields in which the embodiment of invention can be utilized. In the field of dentistry, for example, most of the dental procedures produce an aerosol which can contain pathogens, blood, saliva, tooth/bone fragments which can contaminate the air in a dental operatory for hours, if not contained at the source. The present invention allows dental professionals to more efficiently and effectively control contaminants not only at the source of their creation (e.g., the mouth) but also in a layered manner thereafter in order to create a tiered system of protection from the contaminants for both the patient, the dentist, the dental assistant, the dental hygienist, as well as other dental professionals, other patients, and subsequent patients to be treated in that operatory. Furthermore, a risk exists that contaminants can spread throughout the office through the ventilation system.

### DESCRIPTION OF THE RELATED ART

Using dentistry as an example, the current state of the art for dental hygienists include that they treat their patients by themselves (i.e., without a dental assistant to assist in providing additional suction and retraction). Both dentists and dental hygienists create contaminants when performing almost all their normal functions in the dental office. Dental hygienists often use prophy angles to polish teeth which rotates inside the mouth as well as ultrasonic and piezo scalers which vibrate inside the mouth all of which produce aerosols and contaminants. The dentist often must use a high or low speed drill with a rotating bur or diamond of some sort which places contaminants in the air. These contaminants created by the dentist and dental hygienists enter the air and dental operatory putting both the patient, the dental professionals in the office, and subsequent patients at risk of exposure with said contaminants.

One way in which dental professionals try to increase visibility and patient comfort is to have an assistant using suction when the dentist performs their work Which may help reduce some of the contaminants but not all of them. Unfortunately, since dental hygienist often work alone, they do not have someone there to help them with suction, so the contaminants easily escape the mouth. As soon as these contaminants escape the patient's mouth, they are free to spread and live in the air and on the surfaces of the room which creates a threat to both the dental professionals and patients in the office. Some contaminants are more contagious than others, some live longer in the air and on the surfaces than others, but all of which must be cleared from the room before the next patient enters and the dental professional changes their personal protective equipment so as not to contaminate themselves or others. The act of cleaning a room after it has been exposed to contaminants in such a manner is an arduous, tedious, ineffective and time-consuming process which can allow contaminants to remain and infect dental professionals and or subsequent patients. There is no present or predictable way to effectively sterilize the air once the contaminants have exited the mouth.

### BRIEF DESCRIPTION OF THE FIGURES

For the purpose of illustrating the invention, there is shown in the accompanying drawings forms which are presently preferred; it being understood that the invention is not intended to be limited to the precise arrangements and instrumentalities shown.

FIG. 1 shows a side view of the invention showing three tiers of containment sources;

FIG. 2 shows a front view of the invention from FIG. 1 on a person;

FIG. 3 shows a side view of an embodiment of the invention with a nasal guard on a person;

FIG. 4 shows a front view of a guard with a nasal guard on a person, which is embodiment of the invention;

FIG. 5 shows a side view of a third embodiment of the invention showing several (3) containment sources;

FIG. 6 shows a front view of the third embodiment as shown in FIG. 5 and includes a nasal guard;

FIG. 7 shows a further embodiment as an intraoral device;

FIG. 8 shows examples of embodiments for the field of dentistry including guards that attach to dental devices;

FIG. 9 shows a further embodiment of the invention placed in the mouth and expanded to cover entire surface of the lips;

FIG. 10 is a side view of the embodiment described in FIG. 9;

FIG. 11 is a further embodiment of the invention as a guard incorporated into a dental chair headrest;

FIG. 12 is an even further embodiment of this invention showing a perspective view of a light, chemical, or laser which would be directed at a patient's mouth, and

FIG. 13 shows a further embodiment of a device attached to a dental professional.

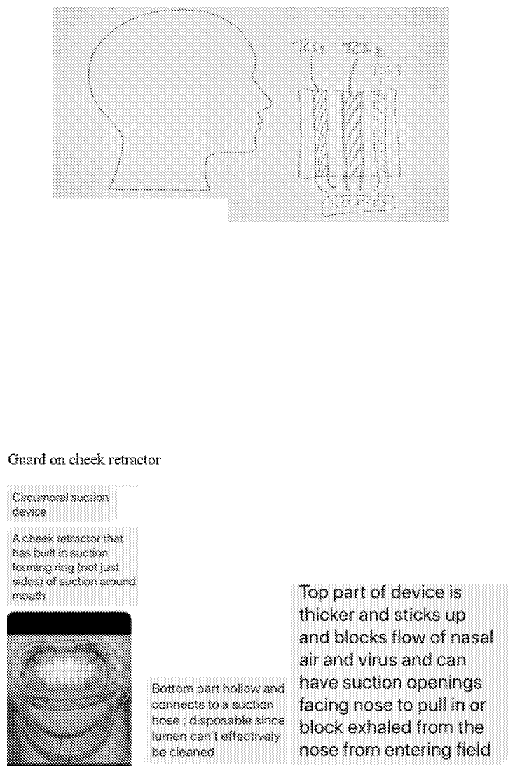

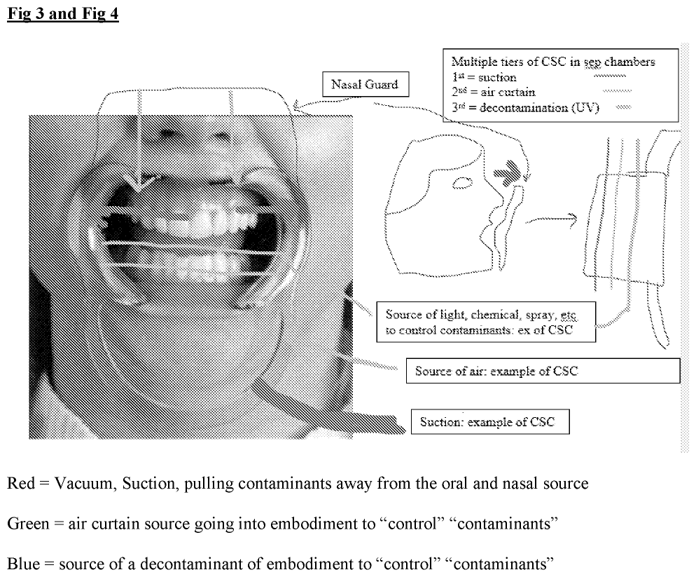

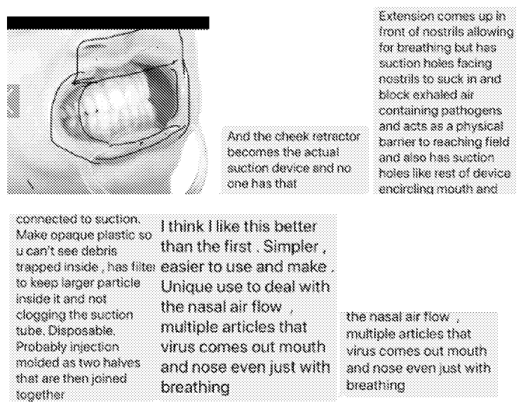

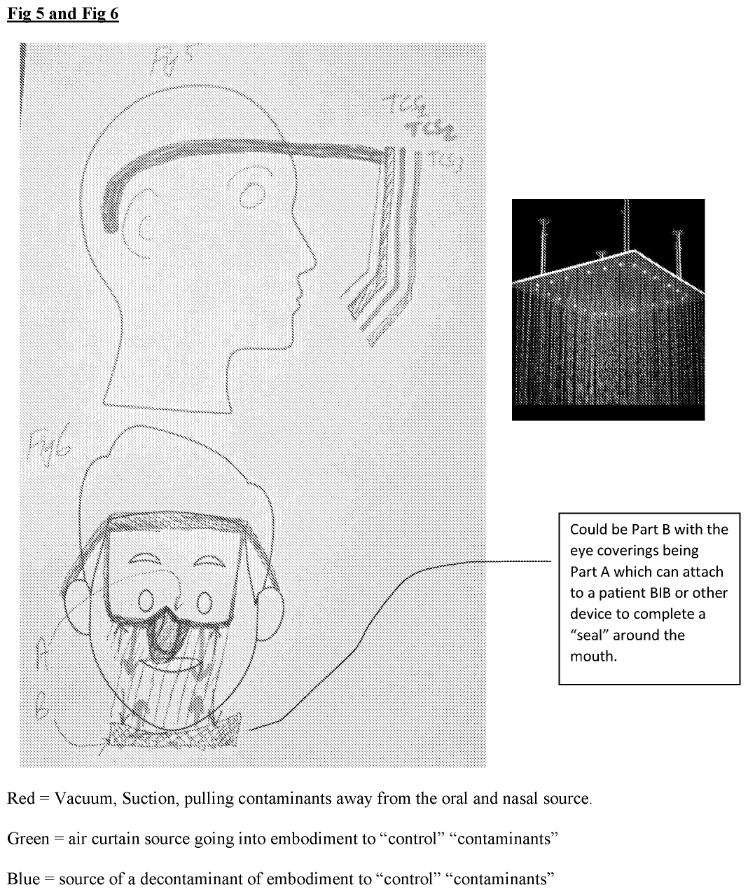

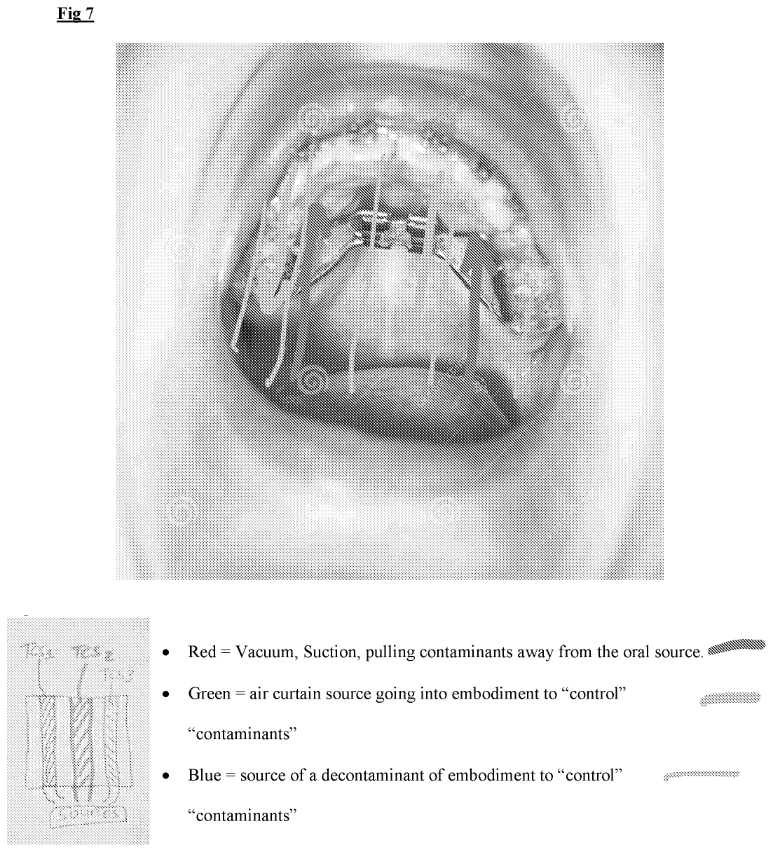

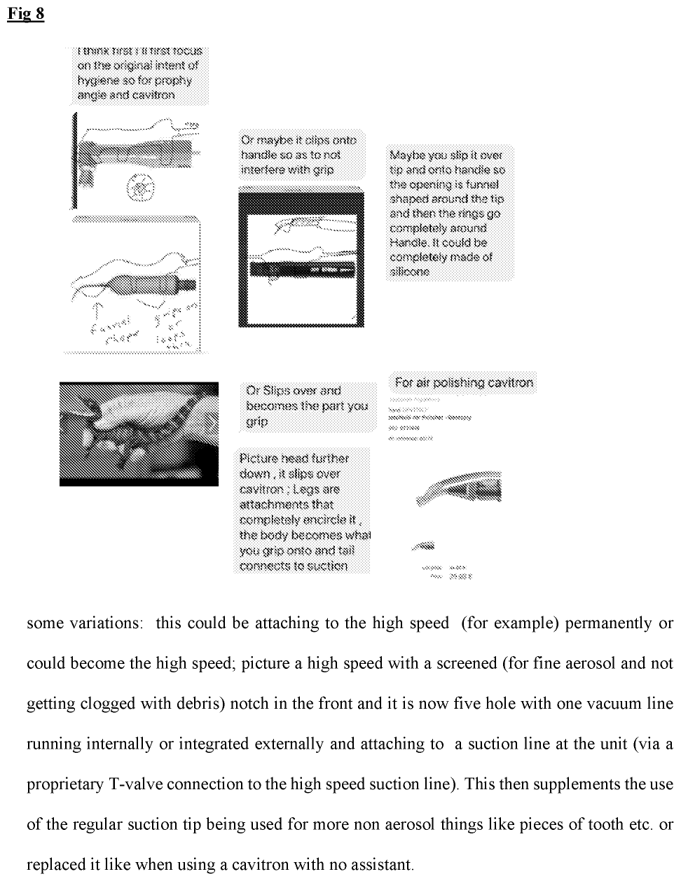

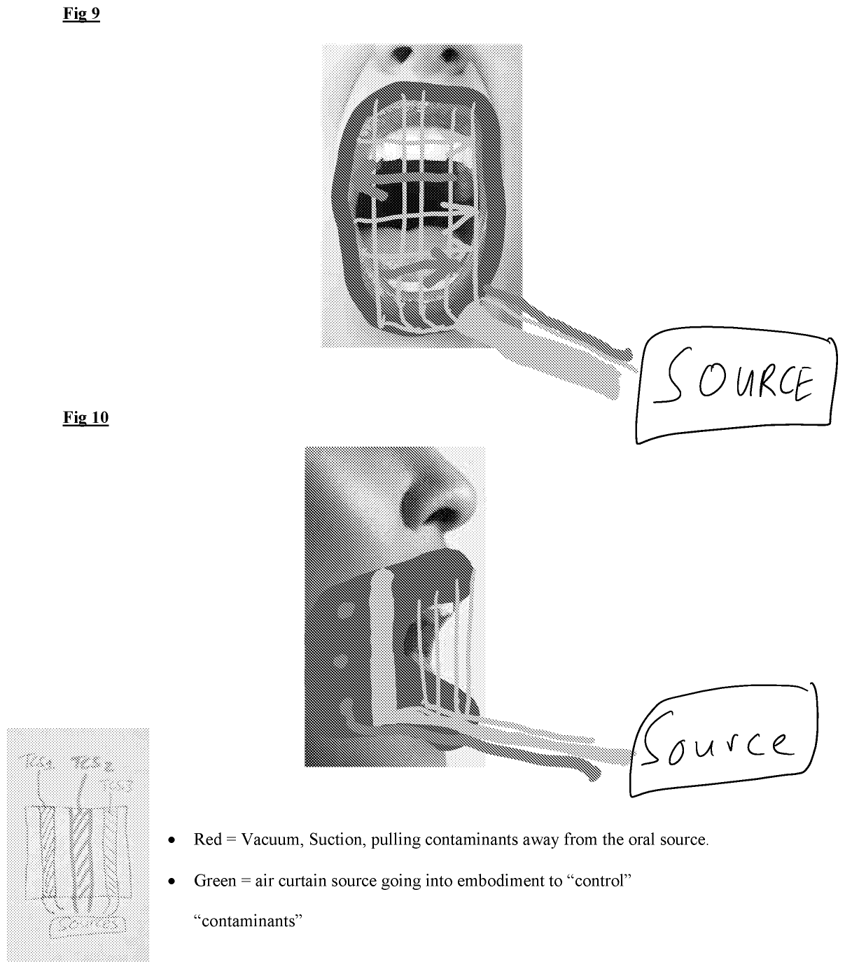

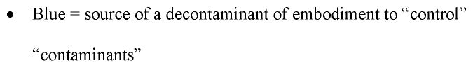

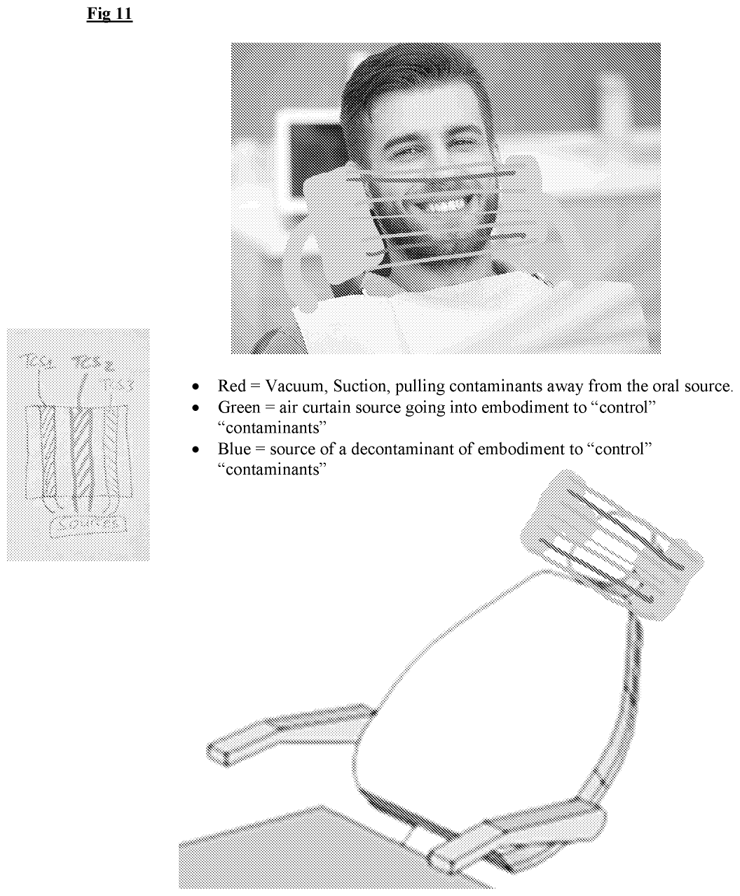

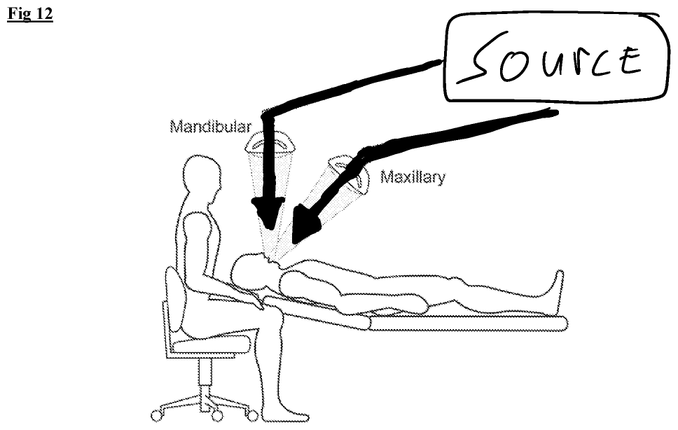

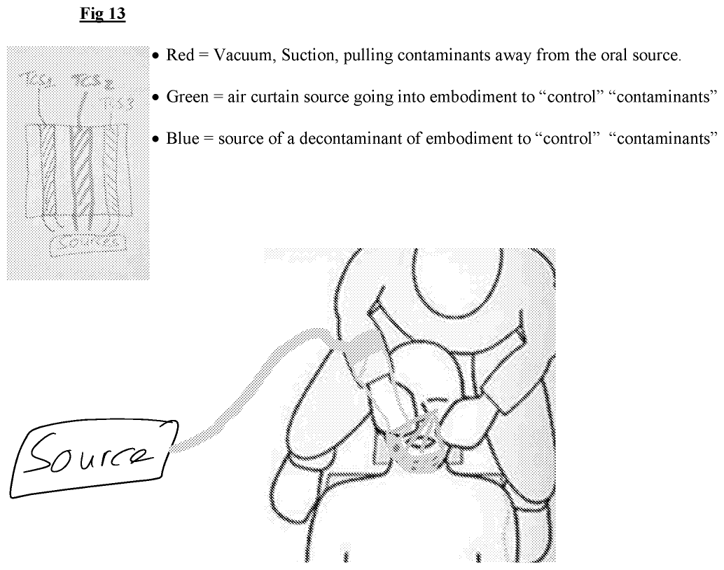

### SUMMARY DESCRIPTION

Embodiments of this invention provide a system, apparatus, and method for reducing, eliminating, neutralizing, deagglomerating, absorbing, suctioning, vacuuming, knocking down, mitigating, etc and/or inactivating/lysing/killing/reducing the amount/concentration (hereafter “control or controlling”) of but not limited to smoke, restorations (amalgam/composite/etc), mercury/chemical vapor, aerosols, droplets, airborne particles, vapors, pathogens, viruses, bacteria, fumes, chemicals, exhaust, splatter, sewage, and body fluids (blood, saliva, bone, teeth, urine, feces, etc.) (hereafter “contaminant”) to which operators, patients, providers, workers, etc are exposed during but not limited to medical, dental, veterinary, automotive, plumbing, residential, professional, commercial, industrial, etc procedures, techniques, services, etc. Embodiments of the invention include a shield, armor, protection, border, or some other configuration (hereafter “guard”) that is capable of accommodating multiple variations and goals of protecting individuals. Embodiments of the invention can be made to accommodate applications of various sizes, configurations, and dimensions. Embodiments of this invention in the field of dentistry for example, are designed to control contaminants in the intra-oral/nasal, and/or peri-oral/nasal, and/or extra-oral/nasal zones (hereafter “contaminant zones” “CZ”) thereby controlling the progression of contaminants from both the oral and nasal airways past the peri-oral/peri-nasal area and infecting surfaces, airways, and/or individuals. Embodiments of this invention utilize tiers to control the contaminants in a short amount of time, distance, etc by layering sources to control the contaminants as swiftly as possible (hereafter “Tier Containment” “TC”). The TC may include tiers of sources that help to contain contaminants such as but not limited to suction, air, chemicals, light, mists, ozone, lasers, etc (hereafter “Tier Decontaminant Method” “TDM”) by pulling, pushing, inactivating, etc contaminants. Embodiments of this invention for example may contain a first tier of suction to pull contaminants out of the air, a second tier that creates an air curtain, and a third tier that decontaminates the air. The suction will first attempt to pull a large portion of contaminants out of the air. The air curtain then provides direction to air movement which creates a barrier which can protect against the movement of contaminants from the intra-oral and intra-nasal cavities into the extra-oral environment further reducing the number of contaminants. The third TDM may be a chemical mist or a UV light that can efficiently treat the vastly reduced number of contaminants that got past the first two tiers to disinfect the air in a shorter amount of time and distance. UV light includes but is not limited to UV-C, and UV-far; or laser or other light source, including mounting in or around or on or within any form of clothing or accessories including but not limited to mask frame, eyeglasses, sunglasses, hat, visor, headband, wallet , handbag, cell phone case, wristband or bracelet , necklace or neckwear, keychain, and/or virtual face mask, etc. The combination of these three TDM thereby reduces the contaminants in the extra-oral/nasal air which reduces the need for additional atmospheric decontamination measures. To adapt embodiments of the invention for multiple purposes, and to standardize the process, embodiments of the invention include adapters that allow for guards and TDMs to be placed under/on top of/into/onto/around, suspended, and/or sealed around an individual to protect them from contaminants. Embodiments eliminate the need for or reduces the expense for large, expensive guards which tend to be at a greater distance from the source of the contaminant. In these embodiments, guards are layered starting at the source of the contaminant and extending away from the source in order to provide an economic, tiered system of protection to best protect individuals from the contaminant. The present invention thus allows crafters, craftsmen, handyman, commercial/industrial workers, mechanics, hobbyists, dental/medical/veterinary and other professionals, etc. to perform actions safety which would otherwise place the individual in the presence of contaminants that could be harmful to them or those around them.

Objects of the invention that pertains to the field of dentistry, for example, include embodiments designed to control contaminants:
as close to the source and/or point of aerosol generation as possible.
in a tiered approach by using multiple guards at various distances from the source.
by using a variety or adjustable amount of air evacuation or other type of evacuation
by utilizing a variety or adjustable amount of air, light, chemicals, or other medium that directs the contaminants into or away from a guard.
by employing the use of light such as but not limited to ultraviolet or laser
by employing the use of a chemical such as but not limited to ozone, an oxidant, etc

1. A system for minimizing risk of airborne contaminants, comprising a guard configuration, where the guard accommodates an at least one variation for controlling contaminants.

2. The system of claim 1, where the guard is applied to an at least one contaminant zone selected from the group of intra-oral/nasal, peri-oral/nasal, and extra-oral/nasal zones.

3. The system of claim 1, where the guard is comprised of an at least one tier containment guard that controls the contaminants for a predetermined length of time and range.

4. The system of claim 3, where the tier containment guard is comprised of tier decontaminant method (TDM), which is comprised of a set of at least two guard applications selected from the group of suction, forced air, chemical treatment, light, misting, ozone, and lasers that pull, push, or inactivate contaminants.

5. The system of claim 1, where a first tier of suction pulls contaminants out of the air, a second tier creates an air curtain, and a third tier that decontaminates the air.

6. The system of claim 5, where the suction pulls a large portion of contaminants out of the air closest to the source of origin, and the air curtain then directs air to create a barrier that protects against the movement of contaminants from the intra-oral and intra-nasal cavities into the extra-oral environment further reducing the number of contaminants.

7. The system of claim 5, where a third tier is comprised of an at least one decontaminant selected from the group of a chemical mist and an at least one UV light.

8. The system of claim 7, where the UV light is selected from the group of UV-C, UV-far, and a laser.

9. The system of claim 1, further comprised of a mounting selected from the group of mask frame, eyeglasses, sunglasses, hat, visor, headband, wallet, handbag, cell phone case, wristband, bracelet, necklace, neckwear, keychain, and a virtual face mask.

10. The system of claim 1 further comprised of adapters that allow for the guard to protect a patient from contaminants.

11. The system of claim 1, further comprised of the steps of decreasing the risk of fomite formation, condensates, cross contamination, and infection, and a step of protecting eyes, skin, and airways by controlling contaminants.

12. The system of claim 11, further comprised of the step of applying ozone.

13. The system of claim 11, where the guard is attached to a dental chair.

14. The system of claim 4, further comprised of a plurality of chambers to push and/or pull TDMs to control the contaminants in the containment zones in an at least one tiered guard system TS.

15. The system of claim 1, where an air curtain directs air movement to create a barrier that protects against the movement of contaminants from the intra-oral and intra-nasal cavities into the extra-oral environment further reducing the number of contaminants.

16. The system of claim 1, where the air curtain wraps around at least a portion of the lips and where the air and suction alternate so that the curtain pushes and pulls air so that contaminants can be removed as close to the source as possible.

17. The system of claim 1, further comprised of a circumferential adaption.

18. The system of claim 1, further comprised of a virtual face mask.

19. The system of claim 1, where the guard is comprised of a suction tier, a forced air tier, and a UV light tier.

20. The system of claim 1, where the guard is comprised of a circumoral suction device, a cheek retractor with a suction forming ring, and where the cheek retractor guard tier is further comprised of a suction hose.
